(19) United States
(12) Patent Application Publication
(10) Pub. No.: US 2017/0131211 A1
(43) Pub. Date: May 11, 2017

(54) SYSTEMS FOR CELL LYSIS AND ANALYTE DETECTION AND ASSOCIATED METHODS

(71) Applicant: University of Washington, Seattle, WA (US)

(72) Inventors: Paul Yager, Seattle, WA (US); Joshua Bishop, Seattle, WA (US); Joshua Buser, Seattle, WA (US); Louise Lyth Hansen, Seattle, WA (US); Erin K. Heiniger, Seattle, WA (US); Enos Kline, Seattle, WA (US); Sujatha Kumar, Seattle, WA (US)

(21) Appl. No.: 15/348,926

(22) Filed: Nov. 10, 2016

Related U.S. Application Data
(60) Provisional application No. 62/253,607, filed on Nov. 10, 2015.

(51) Int. Cl. G01N 21/64; C12Q 1/68

(52) U.S. Cl. CPC G01N 21/6486; C12Q 1/6816

(57) ABSTRACT

The present technology relates generally to systems for disrupting biological samples and associated devices and methods. In some embodiments, the system includes a vessel configured to receive a biological sample and a cap assembly that includes a porous membrane having a receiving region and a detection region. When the cap assembly is detachably coupled to an open end portion of the vessel, the system can be moved between a first orientation and a second orientation. When the system is in the first orientation, the biological sample is not in fluid communication with the receiving region. When the vessel contains is in the second orientation, the biological sample is in fluid communication with the receiving region and wicks through the porous membrane to the detection region.

### CROSS-REFERENCE TO RELATED APPLICATION(S)

The present application claims the benefit of U.S. Provisional Patent Application No. 62/253,607, filed Nov. 10, 2015, which is incorporated by reference herein in its entirety.

### TECHNICAL FIELD

The present technology relates generally to systems and methods for assaying one or more analytes within a biological sample. Many embodiments of the present technology relate to systems and methods for lysing cells and assaying for analytes therein.

### BACKGROUND

Diagnosis is the first hurdle in disease management, enabling expedited appropriate treatment in developed settings where sophisticated equipment and trained personnel are available. For example, in the United States, in-vitro diagnostic procedures represent about 1.6% of Medicare spending, yet influence 60-70% of medical decisions. Nucleic acid amplification tests (NAATs) performed in the laboratory represent the pinnacle of sensitive and specific pathogen detection. Unfortunately, this state of the art is also expensive and complex, requiring infrastructure and instrumentation not available in all settings.

The lack of adequate diagnostics is especially troublesome in the case of tuberculosis (TB), which infects approximately one-third of the world's population according to the World Health Organization (WHO). Sixty percent of TB patients only have access to a peripheral level of the health system, where no suitable TB diagnostics exist. Conventional TB diagnostics in low-resource settings, mainly sputum smear microscopy and cell culture, lack the ideal specificity and timeliness. Also, the required equipment is rarely available.

Microfluidic devices have shown promise to enable the type of point-of-care device that could bring NAATs to the point of care in low-resource settings, but sample preparation, such as cell lysis, remains the weak link in microfluidics-based bioassays. Mechanical lysis methods, such as bead beating, are desirable in that one can avoid the need to purify the sample from a chemical lytic agent before the downstream bioassay, but these methods traditionally suffer from relatively complex, user- and power-intensive instruments and protocols.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a cross-sectional front view of an assay system configured in accordance with an embodiment of the present technology.

FIG. 2A is an isolated, exploded view of a detection assembly of the assay system shown in FIG. 1.

FIG. 2B is an isolated view of the detection assembly of the assay system shown in FIG. 1, shown with the coil removed for ease of illustration.

FIGS. 2C and 2D are side and top views, respectively, of another embodiment of a detection assembly in accordance with an embodiment of the present technology.

FIG. 3 is an isolated view of the tube assembly of the assay system shown in FIG. 1.

FIG. 4 is an isolated, exploded view of the cap assembly of the assay system shown in FIG. 1.

FIGS. 5A-5F illustrate a method for using the assay system shown in FIGS. 1-4 for performing an assay of a biological sample in accordance with an embodiment of the present technology.

### DETAILED DESCRIPTION

The present technology is generally related to systems and methods for assaying one or more analytes of a biological sample and, in some embodiments, to systems and methods for lysing cells and assaying one or more analytes contained within the cells. In certain embodiments of the present technology, the system comprises a cap assembly, a detection assembly having one or more detection units, and a vessel having a closed end portion and an open end portion configured to receive a biological sample. The cap assembly includes a porous membrane having a receiving region and a detection region. When the system is assembled (referred to herein as “the assay assembly”), the detection assembly is positioned around the closed end portion of the vessel and the cap assembly engages and seals the open end portion. In this assembled configuration, the receiving region of the porous membrane is fluidly coupled to an open end portion of the vessel, and the detection region of the porous membrane is positioned adjacent a detection unit of the detection assembly. When the assay assembly is in an upright orientation, the receiving region—which is fluidly coupled to the porous membrane—does not contact and/or is not in fluid communication with the biological sample within the vessel. When the assay assembly is inverted, the biological sample contacts the receiving region and wicks through the porous membrane to the detection region for detection by the detection units.

### I. DEFINITIONS

As used herein, the term “porous membrane” refers to a material through which fluid can travel by capillary action. Representative examples of such porous membranes include glass fiber, paper, nitrocellulose, nylon, cellulose, and many other materials recognized by those skilled in the art as capable of serving as a wick in the context of the present technology. In some embodiments, all or part of the porous membrane may include a cellulose ester or a polymeric material (e.g., polyether sulfone (“PES”), polysulfone (“PS”), polyether sulfone (“PES”), polyacrilonitrile (“PAN”), polyamide, polyimide, polyethylene (“PE”), polypropylene (“PP”), polytetrafluoroethylene (“PTFE”), polyvinylidene fluoride (“PVDF”), polyvinylchloride (“PVC”). The porous membrane can be two-dimensional or three-dimensional (when considering its height in addition to its length and width). In some embodiments, the porous membrane is a single layer, while in other embodiments, the porous membrane comprises two or more layers of membrane.

As used herein, the term “wettably distinct” means being capable of being wetted by contact with separate fluids without mixing of the fluids at the point of initial wetting. For example, two input legs are wettably distinct if they are physically separated so that each leg could be brought into contact with a separate fluid reservoir. Pathways can be made wettably distinct by a variety of means including, but not limited to, separation via distinct edges (e.g., cut as separate pathways) and separation via an impermeable barrier.

As used herein, a “biological sample” can be any solid or fluid sample, living or dead, obtained from, excreted by, or secreted by any living or dead organism, including, without limitation, single-celled organisms, such as bacteria, yeast, protozoans, amoebas, multicellular organisms (such as plants or animals, including samples from a healthy or apparently healthy human subject or a human patient affected by a condition or disease to be diagnosed or investigated, such as tuberculosis) and/or soil. Biological samples can include one or more cells, proteins, nucleic acids, etc., as well as one or more buffers. Biological samples can be a liquid phase solution of cells or it may be a solid cell sample such as a cell pellet derived from a centrifugation procedure. As used herein, a “cell” or “cells” can refer to eukaryotic cells, prokaryotic cells, viruses, endospores or any combination thereof. Cells thus may include bacteria, bacterial spores, fungi, virus particles, single-celled eukaryotic organisms (e.g., protozoans, yeast, etc.), isolated or aggregated cells from multi-cellular organisms (e.g., primary cells, cultured cells, tissues, whole organisms, etc.), or any combination thereof, among others. Furthermore, the term “lysis” or “lyse” as used herein refers to disrupting the structural integrity of a cell (e.g., by breaking the cellular membrane of the cell) in order to gain access to materials within the cell.

Use of relative directional language like top, bottom, upper, lower, up, down, upright, upwards, downwards, and others are relative and are not restricted to absolute directions or orientations defined with respect to the surface of the earth.

### II. SELECTED EMBODIMENTS OF ASSAY SYSTEMS AND METHODS OF USE

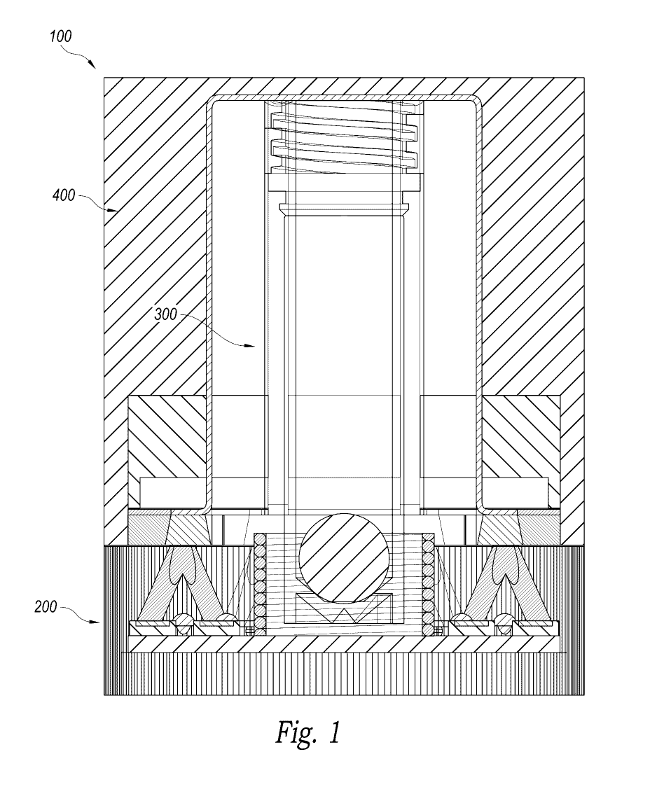

FIG. 1 is a cross-sectional front view of an assembled assay system 100 (also referred to as “system 100”) configured in accordance with an embodiment of the present technology, shown in a first or upright orientation. The system 100 can include a detection assembly 200, a vessel 300, and a cap assembly 400. As described in greater detail below, inversion of the system 100 from the upright orientation to an inverted orientation places the biological sample within the vessel in fluid communication with a porous membrane of the cap assembly, thereby allowing detection of one or more analytes within the biological sample by the detection assembly.

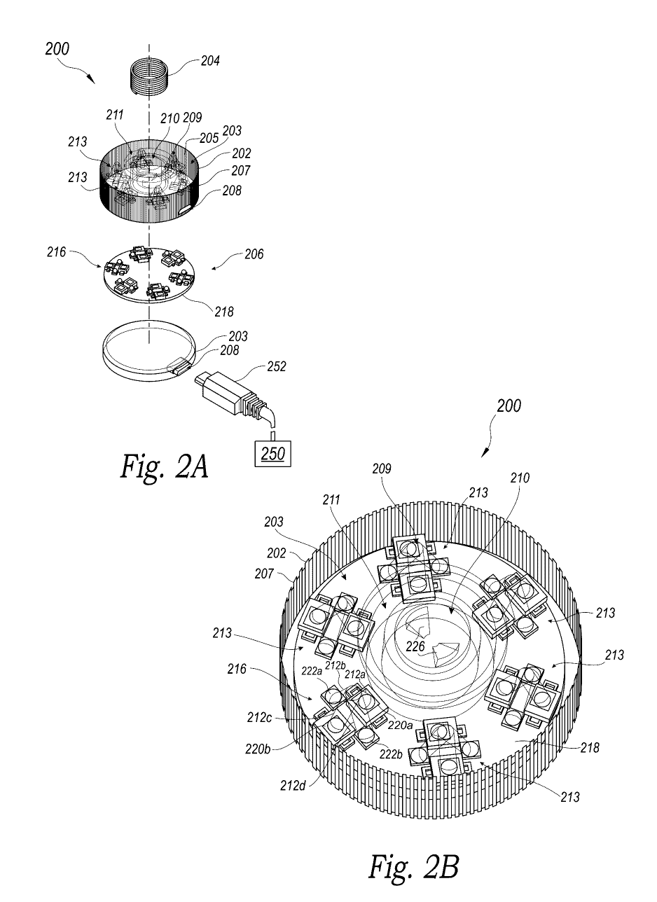

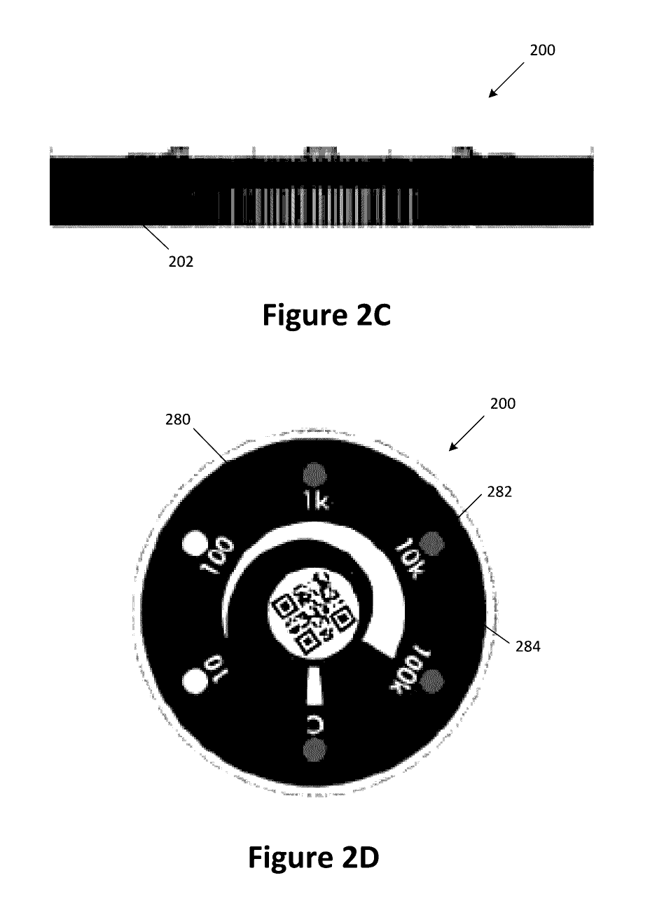

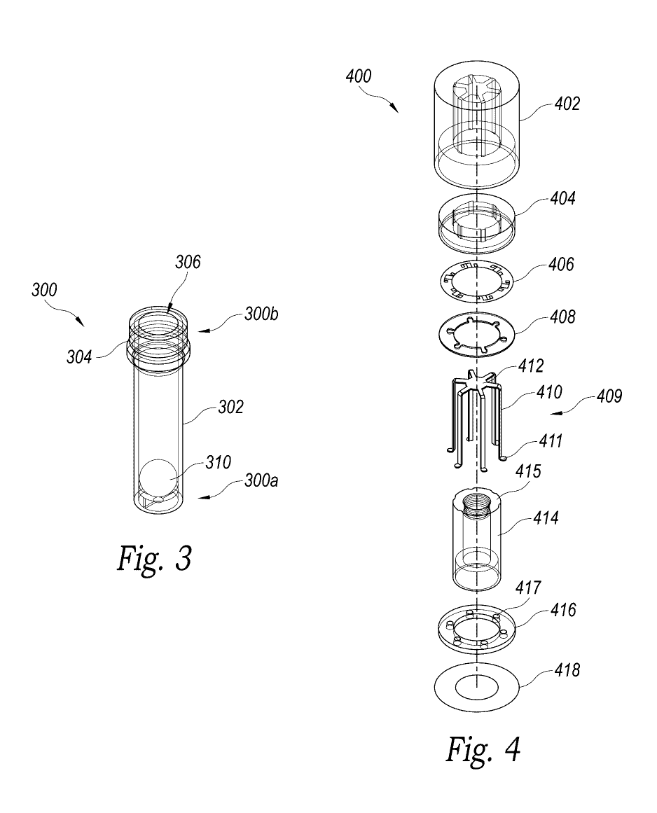

FIG. 2A is an isolated, exploded view of the detection assembly 200 of the system 100, and FIG. 2B is a top view of the detection assembly 200. Referring to FIGS. 2A and 2B together, the detection assembly 200 can comprise a housing 202, an electromagnetic coil 204 (not shown in FIG. 2B for ease of illustration), a detector 206, and a base 203. The housing 202 has a top wall 205 and a sidewall 207 that together define an interior region surrounding the detector 206 and the base 203. Both the top wall 205 and the sidewall 207 are transparent in FIGS. 2A and 2B to better illustrate the interior region 203 of the housing 202 The top wall 205 of the housing 202 includes a plurality of openings 209 positioned around the circumference of the housing 202 (only one labeled in FIG. 2A for ease of illustration), an annular recess 211 radially inward of and spaced apart from the openings 209, and a cavity 210 radially inward of and spaced apart from the annular recess 211. The annular recess 211 is configured to receive a coil 204 of the lysing assembly, as described in greater detail below. The cavity 210 is configured to receive an end portion of the vessel 300, and the housing 202 includes one or more detents 226 extending into the cavity 210 for engaging the vessel 300 to reduce or prevent relative movement between the vessel 300 and the housing 202.

The housing 202 may further include a plurality of waveguides extending downwardly from each of the openings 209 into an interior region of the housing 202. In some embodiments, the housing 202 of the detection assembly 200 is a solid piece of material, and the waveguides are channels that extend through the solid piece of material. In other embodiments, the housing 202 of the detection assembly 200 is generally hollow, and the waveguides are tubes that extend away from the openings 209 and across the interior region 203 of the housing 202.

In the embodiment shown in FIGS. 2A and 2B, the housing 202 includes six sets 213 of waveguides associated with each one of the openings 209 and spaced apart around the circumference of the housing 202. Each of the sets 213 includes four waveguides 212a-212d (referred to collectively as “waveguides 212”) (only one set of waveguides 212 is labeled in FIG. 2B for ease of illustration). In other embodiments, the detection assembly 200 may include more or fewer sets and/or waveguides 212. Within a particular set 213, each of the waveguides 212 has a first end at the corresponding opening 209 and a second end at or near a corresponding detection unit 216 of the detector 2006, as described in greater detail below. The channels defined by the waveguides are optically isolated from one another (other than where the waveguides converge at the detection region 411) such that light waves passing through one waveguide (e.g., 212a) are generally isolated from the light waves passing through another waveguide (e.g., 212c), and vice versa. The openings 209, channels, and/or waveguides 212 may also include one or more filters such that only light of a certain wavelength is allowed to pass through the corresponding waveguide.

Referring still to FIGS. 2A and 2B, the detector 206 includes a printed circuit board (“PCB”) 218 and a plurality of detection units 216 (or “units 216”) positioned on and electrically coupled to the PCB 218. The individual detection units 216 are configured to detect and/or measure one or more analytes in the biological sample, as described in greater detail below. Each of the detection units 216 is aligned with and corresponds to one of the sets 213 of waveguides 212. In the embodiment shown in FIGS. 2A and 2B, the detection units 216 are optical detection units, and each unit 216 includes a first subunit comprising a first photodiode 220a and a first light source 222a (e.g., an LED), and a second subunit comprising a second photodiode 220b and a second light source 222b (e.g., an LED). The first subunit is configured to detect the wavelength of a particular analyte or indicator associated with a particular analyte (e.g., an amplicon). The second subunit is configured to detect the wavelength of a control. A first waveguide 212a extends between an opening 209 and the first photodiode 220a, and a second waveguide 212b extends between the same opening 209 and the first LED 222a. A third waveguide 212c extends between the opening 209 and the second photodiode 220b, and a fourth waveguide 212d extends between the opening 209 and the second LED 222b.

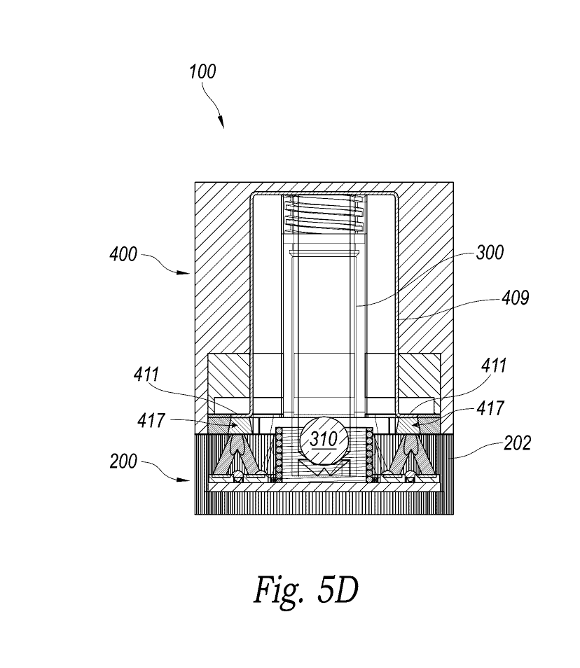

In some embodiments, the detection assembly 200 does not include a PCB (or any chip and/or integrated circuitry) and is configured to visually indicate to the user the presence and/or concentration of a particular analyte in the detection region 411 (discussed in greater detail below with reference to FIG. 4) of the porous membrane 409. For example, as shown in FIGS. 2C and 2D, the detection assembly 200 may include a housing 202 and a pad 280 positioned within the housing 202. The pad 280 includes one or more indication regions 282. When the system 100 is assembled, the detection regions 411 of the porous membrane 409 are placed in direct contact with the indication regions 282 of the pad 280. When the assembled system 100 is inverted, the biological sample travels through the porous membrane 409, to the detection regions 411, and then to the particular indication region 282 associated with and in contact with the corresponding detection region 411. A user may then image the pad 280 (e.g., using an electronic mobile device) to detect the fluorescence at the indication regions 282. The pad 280 may be imaged while part of the assembled system 100 and/or after the user removes the pad 280 from the assembled system 100. In some embodiments, the indication regions 282 may visually indicate the presence and/or amount of a particular analyte without the need for imaging (e.g., the indication regions 282 may change color, etc.).

Each of the indication regions 282 may have a corresponding indicator 284. In the embodiment shown in FIGS. 2C and 2D, the indicators 284 are numbers that represent the maximum amount and/or concentration of a particular analyte (or detection molecule associated with a particular analyte) present in the biological sample at the corresponding detection region 411 (also known as “competitive thresholding”). The assay may be designed such that only a predetermined amount and/or concentration of the analyte will produce a visible fluorescent signal. Thus, in the example provided in FIG. 2D, the biological sample delivered to the porous membrane 209 (before amplification) contained 1,000 copies of a particular nucleic acid, and each of the legs of the porous membrane were impregnated with different, known amounts of amplification reagents. The nucleic acids were amplified as the biological sample moved through the porous membrane 409 and/or when the biological sample reached the detection region 411 and/or indication regions 282 (via one or more amplification reagents present in the porous membrane 409 and/or at the indication regions 282). The indication regions 282 associated with the “1 k”, “10 k”, and “100 k” indicators 284 show a positive fluorescent signal (indicated by the hashed lines) because the number of copies of the particular nucleic acid at those indication regions 282 were greater than or equal to the threshold copy levels of 1,000, 10,000 and 100,000, respectively. Similarly, the indication regions 282 associated with the “10” and “100” indicators 284 show no fluorescent signal because the number of copies of the particular nucleic acid at those indication regions 282 were less than the threshold copy levels of 10 and 100, respectively. The pad 280 may optionally include a control indication region 282.

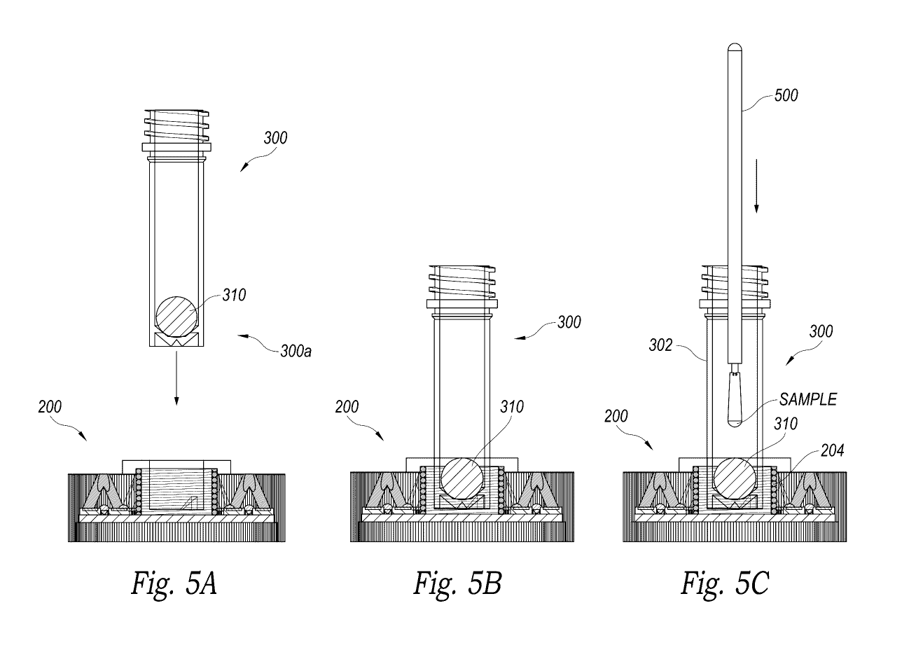

FIG. 3 is an isolated, isometric view of the vessel 300 of the system 100 (FIG. 1). The vessel 300 can be a tube (e.g., a laboratory tube) having a generally cylindrical sidewall 302, an open end portion 300b, and a conical closed end portion 300a. The vessel 300 may, for example, be in the shape of a micro centrifuge tube (e.g., an Eppendorf tube), a centrifuge tube, a vial, etc. As shown in FIG. 3, the vessel 300 can define only one compartment/chamber for holding the biological sample, or a plurality of discrete compartments/chambers (e.g., an array of wells) for holding biological samples in isolation from one another (e.g., a microwell plate, discussed in greater detail below with reference to FIG. 3). The vessel 300 can include an opening 306 at the open end portion 300b for receiving a biological sample to the interior portion of the vessel 300. The vessel 300 can be made of plastic and/or other suitable materials.

It will be appreciated that although the vessel 300 shown in FIGS. 1 and 3 has a generally tubular shape with a conical closed end portion 300a, in other embodiments, the vessel 300 and/or any portion of the vessel 300 can have any suitable size or shape, and/or be made of any suitable material. For example, in some embodiments the closed end portion 300a of the vessel 300 can be rounded (not shown). In a particular embodiment, the vessel 300 has a closed end portion 300a configured to mirror the shape of the agitator 310. For example, in those embodiments where the system 100 (FIG. 1) includes a spherical agitator 310 (as in FIG. 3), the shape of the closed end portion 300a can follow the shape of the spherical agitator 310 (e.g., the vessel 300 can be shaped like a narrow- or wide-necked round-bottom flask).

In order to access certain analytes within the biological sample it may be necessary to lyse or otherwise agitate the biological sample. Accordingly, the systems of the present technology optionally include components or reagents to lyse or otherwise agitate a biological sample. For example, as shown in FIGS. 2A-2B and FIG. 3, the system 100 can include one or more lysing components, such as an agitator 310 (FIG. 3), an electromagnetic coil 204, and a voltage source 250 operably coupled to the electromagnetic coil 204 (e.g., via a cable 252 connected to port 208). As discussed in greater detail below, when the biological sample and agitator 310 are placed within the vessel 300 and the voltage source 250 is activated, the electromagnetic coil 204 produces an alternating magnetic field that causes the agitator 310 to rotate within the vessel 300, thereby lysing at least one of the cells of the biological sample.

The agitator 310 may be pre-loaded in the vessel 300, or the user (not shown) may add the agitator 310 during the assay procedure. The agitator 310 can be generally spherical and configured to be positioned within the vessel 300 adjacent a closed end portion 300a of the vessel 300 when the assembled system 100 is in the upright orientation. In other embodiments, the agitator 310 can have other suitable shapes. For example, in some embodiments, the agitator 310 can be generally cylindrical, disc-shaped, cubical, and/or other suitable polyhedrons and non-polyhedrons. The agitator 310 can be made from a material that is magnetized and creates its own persistent magnetic field, such as a permanent magnet. For example, the agitator 310 can be made from iron, nickel, cobalt, rare-earth metals and some of their alloys (e.g., an Alnico magnet, a neodymium magnet, etc.), naturally occurring minerals such as lodestone, and other suitable materials. As shown in FIG. 3, the agitator 310 can have a diameter that is slightly less than the inner diameter of the vessel 300 such that an outer surface of the agitator 310 is separated from the inner surface of the vessel 300 by a small distance d. The distance d can be small enough to create a region of high shear between the agitator 310 and the interior surface of the vessel 300 when the agitator 310 rotates, but large enough to allow the agitator 310 to rotate freely about any of its plurality of axes, as well as to provide passage for the cells of the biological sample during rotation of the agitator 310. In other embodiments, the agitator 310 can have other suitable sizes, and/or the system 100 can include more than one agitator 310 (e.g., two agitators, three agitators, etc.) and/or one or more agitators configured to translate within the vessel 300 (e.g., bounce around within the vessel 300).

In some embodiments, the system 100 can include one or more lysis reagents capable of chemically lysing a portion of the biological sample. In certain embodiments, the lysis reagents are selected from the group consisting of proteinases (e.g., achromopeptidase, lysostaphin; etc.) salts (e.g., guanidinium thiocyanate), acids, bases, detergents, and buffers.

Referring still to FIGS. 2A and 2B, the electromagnet 204 includes a coiled magnet wire and is positioned within the annular recess 211 of the detection assembly housing 202. The electromagnet 204 is configured to be electrically coupled to the voltage source 250 via a connection 252. Although the port 208 and connection 252 shown in FIGS. 2A and 2B are a USB port and a USB cord, respectively, the system 100 may additionally or alternatively include a port configured to receive an audio cable (e.g., an auxiliary cord). When activated, the electromagnet 204 is provided an alternating current (e.g., an electrical audio signal) via the audio cable. In those embodiments only including a USB port, the USB connection may provide a direct current to the onboard electronics, and the onboard electronics (e.g., an oscillator) converts the direct current to alternating current for delivery to the electromagnet. The connection 252 may be configured to transmit data from the system 100 to an external processor.

The voltage source 250 can be, for example, a battery-powered portable electronic device (e.g., a mobile electronic device) capable of generating an electrical audio signal. For example, the voltage source 250 can include a cell phone, a portable audio device (e.g., a portable mp3 player, a portable radio, a portable cd player, a tape player, etc.), a tablet, a laptop, or other suitable devices.

In some embodiments, the voltage source 250 is configured to deliver a signal having a current of 1 A and an amplitude of 3 V (e.g., with a power of 3 W). In some embodiments, the assembled system 100 may only consume about 100 mW or less. Benchtop power supplies are designed to deliver voltage magnitudes much higher than could be handled by the present system.

The voltage source 250 can be configured to generate and transmit an alternating current that alternates between, for example, about 10 Hz and about 90 Hz (e.g., 30 Hz, etc.). For those embodiments utilizing only a USB connection to the voltage source 250, the system 100 may further include an oscillator (e.g., on the PCB 218) to convert a DC signal to an AC signal. In some embodiments, the voltage source 250 can generate an alternating current that alternates between about 20 Hz and about 60 Hz (e.g., about or equal to 30 Hz, about or equal to 40 Hz, about or equal to 60 Hz, etc.). The voltage source 250 can be connected to the port 208 at the detection assembly 200 via an audio jack, a USB connection, and/or other suitable connections configured to couple to portable electronic devices. In some embodiments, the voltage source 250 can include a display screen (not shown), an electrical output (e.g., an audio jack), and one or more controls. In some embodiments, the display screen is a touch screen. The display screen can indicate to the user various signal parameters, such as the time elapsed, the frequency at which the current is alternating, and the waveform. The voltage source 250 can further include a power button and optional control buttons to adjust one or more of the signal parameters. In some embodiments, the control buttons may be incorporated into a touch-screen display.

The voltage source 250 can further include a processor and memory. The memory can include one or more programs. Each of the programs can include one or more pre-set signal parameters. For example, a first program can output a 30 Hz signal with a sinusoidal waveform, and a second program can output a 40 Hz signal with a square waveform. The programs, however, need not have different values for each parameter. In some embodiments, each of the programs can be tailored to a different lysis procedure. For example, lysis of stronger cells, such as mycobacterium tuberculosis (MTB), may require a higher frequency and/or a longer duration of agitation. As such, the voltage source 250 may contain a program specifically designed for lysis of MTB cells that includes a relatively higher frequency. In some embodiments, one or more programs (e.g., .wav files, .mp3 files, and/or any file that is readable by any device configured to process audio signals) can be downloaded to the voltage source 250 via a hard connection or wirelessly. For example, a frequency and waveform generator application, such as Freq Gen (William Ames), can be downloaded to the voltage source 250 and supply a variety of waveforms at a wide range of frequencies. In some embodiments, the system 100 can further include an amplifier (not shown) to increase the power delivered by the voltage source 250.

In some embodiments, the processor and/or memory may include a program that is configured to generate a signal of varying frequency (e.g. sweep from about 10 Hz to about 150 Hz) and/or complicated wave shapes (e.g., a 30 Hz signal overlaid on a 31 Hz).

Additional details regarding devices, systems and methods for disrupting biological samples for use with the assay systems of the present technology can be found in U.S. patent application Ser. No. 14/601,966, filed Jan. 21, 2015, U.S. Provisional Patent Application No. 61/929,769, filed Jan. 21, 2014, and Buser et al., “Lab on a Chip”, 15, 1994-1997 (2015), each of which is incorporated herein by reference in its entirety.

FIG. 4 is an isolated, exploded view of the cap assembly 400 of the system 100 (FIG. 1). The cap assembly includes an outer housing 402, a first insulation element 404, a thin film heater 406, a second insulation element 408, a porous membrane 409, an inner housing 414, a cold plate 416, and a plastic seal 418. In other embodiments, the cap assembly 400 can have more or fewer components and/or can have other configurations.

Referring to FIGS. 3 and 4 together, the cap assembly 400 is configured to detachably couple to the open end portion 300b of the vessel 300 to seal the open end portion 300a and position a portion of the porous membrane 409 such that, when the assembled system 100 is inverted, the portion of the porous membrane as the open end portion 300a is placed in fluid communication with the biological sample. As used herein, “sealing” refers to substantially confining a biological sample or other fluid to the vessel such that, when the vessel 300 is inverted, the biological sample does not leak. Sealing can be accomplished by removably or permanently affixing the cap assembly 400 to the open end portion 300b of the vessel 300. The cap assembly 400 can be affixed to the vessel 400 with a number of commonly known and used components, such as threads 415 of the inner housing 414 and threads 304 of the vessel 300, adhesives, a friction fit, and/or pressure applied by a user.

The porous membrane 409 has a receiving region 409 configured to be positioned at the open end portion of the vessel 300 when the system 100 is assembled. In the embodiment shown in FIG. 4, the porous membrane 409 includes six wettably distinct legs 410 branching from the receiving region 412 and terminating at a detection region 411. In other embodiments, the porous membrane 409 may have more or fewer than six legs (e.g., no legs, one leg, two legs, three legs, four legs, five legs, seven legs, eight legs, etc.) For example, a porous membrane having no legs may be used for drying the sample for transfer to storage or another assay system and/or for later analysis. In some embodiments, the legs 410 can have increasing levels of nucleic acid molecules complementary to the primer nucleic acid molecules. The detection reagents on each of the legs 410 are configured to indicate the presence of the target analyte with an intensity that is inversely proportional to the concentration of the number of nucleic acid molecules and/or the number of nucleic acid molecules complementary to the primer nucleic acid molecules. Accordingly, several embodiments of the assay systems of the present technology can provide quantitative analyte measurements. In some embodiments of the present technology, the system 100 and/or porous membrane 409 may additionally or alternatively include one or more non-visible detection reagents. For example, in some embodiments, the non-visible detection reagents may be detected by a separate component. In some embodiments, the detection reagents may be visible, non-fluorescent reagents (e.g., colorimetric indicators such as gold nanoparticles, solution turbidity, etc.)

One or more portions of the porous membrane 409 (e.g., receiving region 412, legs 410, and/or detection region 411) may optionally include one or more detection reagents configured to bind to a particular analyte and/or a molecule associated with a particular analyte that indicates the presence and/or amount of the analyte to a user. In one variation of this embodiment, each of the legs 410 includes a different detection reagent configured to detect and/or specifically bind to different analytes. For example, in some embodiments, the detection reagents are fluorescent detection reagents.

In certain embodiments, one or more portions of the porous membrane may have nucleic acid amplification reagents impregnated therein. Nucleic acid amplification reagents may be selected from the group consisting of primers, probes, polymerases, enzymes, deoxynucleoside triphosphate (“dNTP”), nucleic acid control targets, salts, detergents, reducing agents, buffers, glycerol, reagents enabling dry preservation including sugars (e.g., trehalose, dextran, etc.), polyethylene glycol, and others. In certain embodiments the nucleic acid amplification reagents are configured to perform loop-mediated isothermal amplification (“LAMP”), strand displacement amplification (“SDA”), isothermal strand displacement amplification (“iSDA”), recombinase polymerase amplification (“RPA”), and other suitable isothermal nucleic acid amplification reactions. In some embodiments, one or more portions of the porous membrane may additionally or alternatively have protein capture and detection reagents impregnated therein.

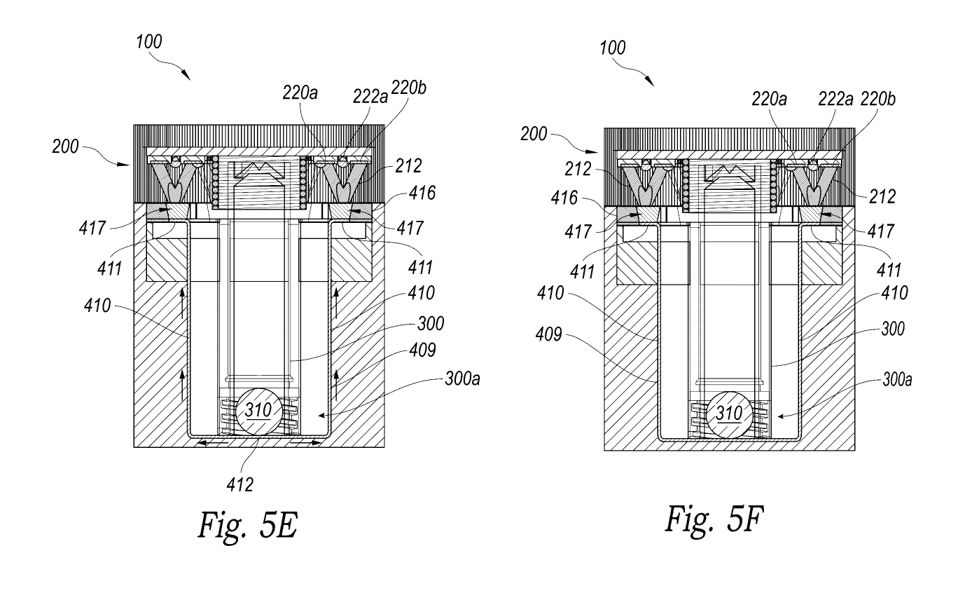

FIGS. 5A-5F illustrate different stages of a method for lysing and assaying one or more analytes of a biological sample utilizing the assay systems of the present technology. As shown in FIGS. 5A and 5B, the vessel 300 can be initially positioned within the detection assembly 200 such that the closed end portion 300b of the vessel 300 is surrounded by the electromagnetic coil 204. In other embodiments, however, the vessel 300 may come pre-assembled with the detection assembly 200. Referring next to FIG. 5C, a user (not shown) can deliver a biological sample to an interior portion of the vessel 300 (for example, using a swab 500). The biological sample may include one or more analytes therein. As shown in FIG. 5D, the cap assembly 400 can be detachably coupled to the open end portion 300a of the vessel. When the cap assembly 400 is coupled to the vessel 300, the detection regions 411 of the porous membrane 409 are aligned with the openings 209 (not labeled in FIG. 5D) in the detection assembly 200 housing 202. In some embodiments, the cap assembly 400 includes openings 417 that optically couples the detection regions 411 with the openings 209.

Before and/or after coupling the cap assembly 400 to the vessel 300, the electromagnetic coil 204 may optionally receive a current from a voltage source, thereby causing the agitator 310 in the vessel to move (e.g., rotate) and lyse one or more cells within the biological sample. Additionally or alternatively, the system 100 may include one or more lysis reagents to lyse the cells of the biological sample and/or a heating element (described in greater detail below) to aid in lysing the cells of the biological sample.

Referring next to FIG. 5E, the assembled system 100 may be inverted such that gravity pulls the biological sample to the open end portion 300a of the vessel 300 where the biological sample contacts the receiving region 412 of the porous membrane 409. The biological sample wicks upwardly along the legs 410 of the porous membrane 409 to the corresponding detection regions 411. As shown in FIG. 5F, the LEDs of the individual detection units 216 emit light that travels through the corresponding waveguide 212 and activates one or more detection reagents in the biological sample. The photodiode associated with the LED detects the fluorescence of the biological sample and communicates a signal characterizing the fluorescence to the PCB 418 and/or a processor in communication with the photodiode (e.g., a processor associated with a mobile electronic device). Thus, the system 100 detects the presence and/or concentration of one or more analytes of the biological sample at the detection region.

Several embodiments of the present technology enable competitive, quantitative measurements of nucleic acid analyte molecules within the biological sample. For example, in those embodiments in which the porous membrane 406 includes two or more wettably distinct legs 410, the legs may be impregnated with nucleic acid amplification reagents. Each of the porous membranes can comprise nucleic acid molecules that are complementary to the primer nucleic acid molecules. In certain further embodiments, each of the wettably distinct legs contain different numbers or concentrations of nucleic acid molecules complementary to the primer nucleic acid molecules. The primer nucleic acid molecules and the nucleic acid molecules complementary to the primers can form dimers and thereby inhibit nucleic acid amplification. If roughly the same number of analyte nucleic acid molecules enters each of the wettably distinct legs, and each of the legs contain different numbers or concentrations of the nucleic acid molecules complementary to the primer nucleic acid molecules, then the nucleic acid amplification reactions in each of the wettably distinct legs will be inhibited to variable and known extents.

In certain embodiments, the system 100 and/or cap assembly 400 can include a distributing porous membrane having a first end and a second end and varying widths between the first and second ends, wherein the first porous membrane is in fluidic communication with a first portion of the distributing porous membrane having a first width and the at least second porous membrane is in fluidic communication with a second portion the distributing porous membrane having a second width, and wherein the first width and the second width are different. When the system 100 is flipped from a first orientation to a second orientation, the biological sample is placed in fluid communication with the distributing porous membrane. The biological sample is then wicked through the distributing porous membrane to the other porous membranes. Since the first and second porous membranes have overlapping intersections with the distributing porous membrane of varying areas, the first and second porous membranes will receive varying volumes of fluid from the biological sample. Thus, the distributing porous membrane acts as a volume metering element, automatically metering out different volumes of fluid from the biological sample.

Any of the assay systems disclosed herein can optionally include one or more heating units configured to heat one or more portions of the system. For example, the heating unit may be positioned or otherwise configured to heat at least a portion of the porous membrane, at least a portion of the vessel, and/or at least a portion of the biological sample. Heating one or more portions of the system, such as the vessel and/or the biological sample, may be beneficial for assisting lysing of the biological sample and/or deactivating certain lysis reagents, such as achromopeptidase. The application of heat to the porous membrane may assist in nucleic acid amplification reactions, such as isothermal nucleic acid amplification reactions.

The heating unit(s) may be coupled to the detection assembly 200, the vessel 300, and/or the cap assembly 400. In certain embodiments, the heating unit includes an electrical heating unit that is powered by a voltage source (e.g., voltage source 250 shown in FIG. 2A). In some embodiments, the heating unit includes a chemical heating unit that utilizes a chemical reaction to generate heat. Such chemical heating units can be activated and/or powered by a chemical reaction between two or more reagents, such as MgFe and saline. In a particular embodiment, the system includes an electrical heating unit and a separate, chemical heating unit. In another embodiment, the system includes two electrical heating units, and in yet another embodiment, the system includes two chemical heating units.

### III. CONCLUSION

From the foregoing, it will be appreciated that specific embodiments of the technology have been described herein for purposes of illustration, but that various modifications may be made without deviating from the technology. For example, although many of the embodiments are described above with respect to devices, systems, and methods for lysing cells and/or assaying for analytes contained therein, other embodiments are within the scope of the present technology. For example, devices, systems, and methods of the present technology can be used to disrupt (e.g., mechanically, electrically, and/or chemically) or agitate any non-cellular biological sample (e.g., mucus) and/or non-cellular components of the biological sample.

Additionally, other embodiments of the present technology can have different configurations, components, and/or procedures than those described herein. For example, other embodiments can include additional elements and features beyond those described herein, or other embodiments may not include several of the elements and features shown and described herein. For example, in some embodiments the system 100 does not include the coil and/or agitator and is not configured for connection to a current or power source. In such embodiments, the cells of the biological sample may be lysed prior to delivery to the vessel 300. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A system for assaying a biological sample, the system comprising:
a vessel having a closed end portion and an open end portion, wherein the vessel is configured to receive a biological sample; and
a cap assembly including a porous membrane having a receiving region and a detection region, wherein the cap assembly includes a coupling element configured to be detachably coupled to the open end portion of the vessel,
wherein, when the cap assembly is detachably coupled to the open end portion of the vessel via the coupling element, the system has (a) a first orientation in which the open end portion of the vessel faces in a first direction and the closed end portion of the vessel faces in a second direction, and (b) a second orientation in which the open end portion of the vessel faces in the second direction and the closed end portion faces in the first direction, and
wherein—
when the vessel contains the biological sample and the system is in the first orientation, the biological sample is not in fluid communication with the receiving region, and
when the vessel contains the biological sample and the system is in the second orientation, the biological sample is in fluid communication with the receiving region and wicks through the porous membrane to the detection region.

2. The system of claim 1 wherein the system, when in the second orientation, is substantially parallel and opposite to the arrangement of the system in the first orientation.

3. The system of claim 1, further comprising a detection assembly having a detection housing and a detection unit within the detection housing, wherein the detection unit is configured to measure a fluorescence intensity associated with one or more analytes within the biological sample, and wherein the detection housing defines a cavity that is configured to receive the closed end portion of the vessel.

4. The system of claim 3 wherein the detection unit is configured to communicate the measured fluorescence intensity to a user, and wherein the fluorescence intensity is related to an amount of the one or more analytes present within the biological sample.

5. The system of claim 3 wherein, when the cap assembly is detachably coupled to the vessel, a portion of the cap assembly is positioned adjacent the detection region of the porous membrane such that the detection region is aligned with the detection unit of the detection assembly.

6. The system of claim 3 wherein the detection unit includes a photodiode for measuring fluorescence, the photodiode configured to be electrically coupled to a processor.

7. The system of claim 3 wherein the detection unit is configured to provide a visual indication to a user that is proportional to an amount of the one or more analytes present within the biological sample.

8. The system of claim 3 wherein the cap assembly includes a cap housing having a first end portion and a second end portion opposite the first end portion, and wherein, when the cap assembly is detachably coupled to the vessel, the first end portion is positioned adjacent the open end portion of the vessel and the second end portion is positioned adjacent the detection assembly.

9. The system of claim 1 wherein the porous membrane is impregnated with one or more detection reagents.

10. The system of claim 9 wherein the one or more detection reagents are fluorescent detection reagents and the system further comprises a light source configured to excite the fluorescent detection reagents.

11. The system of claim 1 wherein, when the vessel contains the biological sample and the system is in the first orientation, the biological sample is fluidly coupled to but not in fluid communication with the receiving region.

12. The system of claim 1, further comprising a lysing assembly configured to lyse one or more cells in the biological sample while the biological sample is within the vessel.

13. The system of claim 12 wherein the lysing assembly comprises:
a permanent magnet configured to be positioned within the vessel; and
an electromagnetic coil configured to be positioned proximate the vessel and operably coupled to a voltage source, wherein the voltage source is configured transmit alternating current to the electromagnet coil,
wherein, when the biological sample is placed within the vessel and the alternating current is transmitted to the electromagnetic coil, the electromagnetic coil produces an alternating magnetic field that causes the permanent magnet to rotate within the vessel, thereby disrupting at least a portion of the biological sample.

14. The system of claim 12 wherein the lysing assembly is a component of the detection assembly.

15. The system of claim 1, further comprising at least one of a heating element and a lysing assembly, wherein the system is configured to be coupled to a power source for powering at least one of the lysing assembly and the heating element.

16. The system of claim 15 wherein the power source is the audio jack of a mobile electronic device.

17. The system of claim 15 wherein the system includes a port and is configured to be electronically coupled to the power source via a USB connection.

18. The system of claim 15 wherein the power source is a battery.

19. The system of claim 1, further comprising a heating element configured to heat the biological sample while it is positioned at or within at least one of the vessel and the porous membrane.

20. The system of claim 19 wherein the heating element is configured to generate heat via a chemical reaction at the heating element.

21. The system of claim 1, further comprising a buffer comprising lysis reagents for lysing the biological sample.

22. The system of claim 21 wherein the lysis reagents are selected from the group consisting of proteinases, salts, acids, bases, detergents, and buffers.

23. The system of claim 21 wherein the lysis reagents include proteinases, and wherein the proteinases include at least one of achromopeptidase and lysostaphin.

24. The system of claim 21 wherein the lysis reagents include salts, and wherein the salts include guanidinium thiocyanate.

25. The system of claim 1 wherein the porous membrane is impregnated with nucleic acid amplification reagents.

26. The system of claim 25 wherein the nucleic acid amplification reagents include at least one of primers, probes, polymerases, enzymes, deoxynucleoside triphosphate (“dNTP's”), nucleic acid control targets, salts, detergents, reducing agents, buffers, glycerol, reagents enabling dry preservation, sugars, and polyethylene glycol.

27. The system of claim 1 wherein the porous membrane is a first porous membrane and the cap assembly includes a second porous membrane that is wettably distinct from the first porous membrane, and wherein, when the cap assembly is detachably coupled to the open end portion of the vessel and the system is in the first orientation, the biological sample in the vessel wicks into the first porous membrane and the second porous membrane.

28. The system of claim 27 wherein the first porous membrane and the second porous membrane include different amounts of nucleic acid molecules.

29. A system for assaying a biological sample, the system comprising:
a vessel having a closed end portion and an open end portion, wherein the vessel is configured to receive a biological sample;
a cap assembly including a porous membrane having a receiving region and a detection region, wherein the cap assembly includes a coupling element configured to detachably couple and seal the open end portion of the vessel; and
a detection assembly having a housing and a detection unit within the housing, wherein the detection unit is configured to measure a fluorescence intensity associated with one or more analytes within the biological sample, and wherein the detection housing defines a cavity that is configured to receive the closed end portion of the vessel,
wherein, when the cap assembly is detachably coupled to the open end portion of the vessel and the closed end portion of the vessel is positioned within the cavity, inversion of the system places the biological sample in fluid communication with the receiving region of the porous membrane, thereby causing the biological sample to wick through the porous membrane to the detection region for detection of the one or more analytes within the biological sample.

30. The system of claim 29 wherein the detection assembly further includes a lysing assembly.

31. A method of detecting the presence of one or more analytes in a biological sample, the method comprising:
delivering a biological sample into a vessel, wherein the biological sample includes one or more analytes;
detachably coupling a cap assembly to an open end portion of the vessel, thereby forming an assay assembly, wherein the cap assembly includes a porous membrane having a receiving region and a detection region;
inverting the assay assembly to place the biological sample in fluid communication with a receiving region of the porous membrane;
wicking the biological sample along the porous membrane from the receiving region to the detection region; and
detecting the presence of the one or more analytes based on the biological sample at the detection region.

32. The method of claim 31, further comprising lysing at least a portion of the biological sample before placing the biological sample in fluid communication with the receiving region of the porous membrane.

33. The method of claim 32 wherein lysing at least a portion of the biological sample includes mechanically agitating one or more cells within the biological sample with an agitating element powered by an audio jack of a mobile electronic device.

34. The method of claim 31, further comprising heating at least a portion of the biological sample before placing the biological sample in fluid communication with the receiving region of the porous membrane.

35. The method of claim 31 wherein detecting the presence of the one or more analytes includes measuring a fluorescence of the biological sample at the detection region after the assay assembly has been inverted.
